(19) United States
(12) Patent Application Publication
(10) Pub. No.: US 2009/0305954 A1
(43) Pub. Date: Dec. 10, 2009

(54) HISTIDINE-CONTAINING DIASTEREOMERIC PEPTIDES AND USES THEREOF

(75) Inventors: Yechiel Shai, Yahud (IL); Arik Makovitzki, Mazkeret Batya (IL)

Correspondence Address: BROWDY AND NEIMARK, P.L.L.C.;624 NINTH STREET, NW, SUITE 300, WASHINGTON, DC, 20001-5303, US

(73) Assignee: Yeda Research and Development Co. Ltd., Rehovot (IL)

(21) Appl. No.: 12/159,598

(22) PCT Filed: Dec. 27, 2006
(86) PCT No.: PCT/IL2006/001496

§ 371 Date: Jun. 27, 2008

Related U.S. Application Data
(60) Provisional application No. 60/753,401, filed on Dec. 27, 2005.

(51) Int. Cl. A61K 38/12; C07K 7/08; C07K 7/64; A61K 38/10; C12N 5/02; A23L 3/3463

(52) U.S. Cl. 514/9; 530/327; 530/326; 530/317; 514/14; 514/13; 435/404; 426/335

(57) ABSTRACT

Diastereomeric peptides with a net positive charge greater than +1, and cyclic derivatives thereof, are provided, having at least 13 amino acid residues, comprising histidine and one or more hydrophobic amino acid residues, optionally esterified or amidated at the C-terminus and/or acylated at the N-terminus. The peptides may contain other amino acid residues including non-natural amino acids. The peptides are particularly useful in the treatment of cancer.

### FIELD OF THE INVENTION

The present invention relates to histidine-containing diastereomeric peptides and to pharmaceutical compositions comprising them.

### BACKGROUND OF THE INVENTION

Chemical anti-cancer agents are non-specific and consequently damage healthy tissues as well. Therefore, despite the reported advances in early detection and the aggressive treatment of the disease in its initial stage, the overall mortality rate does not appear to have fallen. This has stimulated the search for new drugs with new modes of action and the potential to overcome the inherent resistance.

One approach is to develop polypeptides that control apoptosis. An alternative approach is to use cell lytic peptides. In light of this, several studies reported that antimicrobial peptides, a subgroup that belongs to a large family of cytolytic peptides, act in vitro against different types of cancer cells (Baker et al. 1993; Ellerby et al., 1999; Chen et al., 2001; Mai et al., 2001). These peptides are known to have a central role in the innate immunity of all organisms, including insects, amphibians, and mammals (Boman, 1995). Examples include human defensins (Biragyn et al., 2001; Yang et al., 2002; Oppenheim et al., 2003), cecropins (Hui et al., 2002), cecropin-magainin hybrids (Shin et al, 2001; Park et al., 2003), magainins (Baker et al., 1993), peptides conjugated to homing domains (Ellerby et al., 1999; Chen et al., 2001; Mai et al., 2001), propeptides (Warren et al., 2001) and others (Leuschner et al., 2003; Wang and Wang, 2004). These peptides preferentially bind and disrupt negatively charged phospholipid membranes, the major component of bacterial cytoplasmic membrane. However, it is not clear why some of them bind better and kill several types of cancer cells compared with normal cells (Chan et al., 1998; Papo and Shai, 2003).

Despite the potent anticancer activity of these peptides in vitro, studies in vivo regarding the use of native, all-L amino acid antimicrobial peptides have been very limited, mainly due to the loss of their activity in serum, partially because of their binding to serum components and their enzymatic degradation. These studies include (i) apoptotic peptides conjugated to homing domains that were targeted to specific tissues (Ellerby et al., 1999; Chen et al., 2001) and (ii) intraperitoneally (i.p.) injected antimicrobial peptides derived from magainin and its all D-amino acid analog against ovarian cancer (Baker et al., 1993).

The present inventors have shown previously that introduction of D-amino acids into non-cell-selective lytic peptides resulted in diastereomeric peptides that had selective killing activity toward cells, which are enriched with negatively charged phospholipids in their outer surface (Oren and Shai, 1996). Moreover, these peptides were shown to be potent toward bacteria, and some of them were active also toward cancer cells (Papo and Shai, 2003). Most importantly, this family of peptides preserved their activity in serum and their enzymatic degradation could be controlled. One of these peptides, a 15-amino acid diastereomer composed of leucine, arginine and lysine residues, was recently shown to be active against mouse melanoma and lung carcinoma cell lines, and to significantly inhibit lung metastasis formation in mice with no detectable side effects (Papo et al., 2003)

PCT Publications WO 97/31019, WO 98/37090 and WO 02/040529, of the same applicant, describe peptides comprising both L- and D-amino acid residues with a net positive charge greater than +1. WO 98/37090 describes non-natural synthetic peptides composed of varying ratios of at least one hydrophobic amino acid and at least one positively charged amino acid, in which sequence at least one of the amino acid residues is a D-amino acid. Several diastereomers comprising from 6 to 30 amino acid residues are disclosed in WO 98/37090, but the biological activity was tested only for some 6-mer, 8-mer and 12-mer peptides. Some short-model peptides (12-amino acid long) composed of only leucine and lysine at varying ratios, in which one-third of the sequence consisted of D-amino acids, were further investigated and some of them were found to have reduced hemolytic activity (Hong et al., 1999; Oren et al., 1997).

WO 02/040529 describes peptides having at least 15 amino acids residues, composed of varying ratios of the hydrophobic amino acid leucine, the positively charged amino acid lysine and optionally arginine. Some of the peptides were shown to exhibit antibacterial, antifungal, anti-mycoplasma, and anticancer activity. However, the peptide exhibiting anticancer activity was shown to be toxic to the animals tested already at concentrations which are mildly higher (30%-100%) than those used for treatment of cancer and could be administered at higher and more effective concentrations only when encapsulated in liposomes.

Several publications by the inventors (Malina and Shai, 2005; Avrahami and Shai, 2004; Avrahami and Shai, 2003) disclose that the attachment of aliphatic acids with different lengths (10μ, 12, 14 or 16 carbon atoms) to the N-terminus of a biologically inactive cationic peptide containing both D- and L-amino acids is sufficient to endow the resulting lipopeptide with lytic activity against different cells. WO 2004/110341 discloses such lipophilic conjugates comprising a peptide coupled to a fatty acid, wherein the peptide has at most 12 amino acid residues and may contain histidine residues and D-amino acid residues.

### SUMMARY OF THE INVENTION

The present invention relates to a diastereomeric peptide with a net positive charge greater than +1, and cyclic derivatives thereof, having at least 13 amino acid residues, comprising histidine and one or more hydrophobic amino acid residues, optionally esterified or amidated at the C-terminus and/or acylated at the N-terminus, with exclusion of the peptides set forth in SEQ ID NOs: 45 to 52.

The diastereomeric peptides of the invention contain preferably 13, 14 and more preferably 15 or 16 amino acid residues, or 17 amino acid residues for the cyclic peptides. They may comprise any number of histidine residues, for example, from 1 to 10. Any of the amino acid residues within the sequence may be a D-amino acid residue, either the His or another residue. The number of D-amino acid residues may vary and may be from 1 to 10, preferably 3, 7, 9, and more preferably, five acid residues within the sequence are D-amino acid residues (one third of the residues when the peptide is a 15-mer).

The one or more hydrophobic amino acid residues may be derived from naturally or non-naturally occurring hydrophobic amino acids. In one preferred embodiment, the hydrophobic amino acid residues are from naturally occurring α-amino acids selected from alanine, cysteine, isoleucine, leucine, methionine, phenylalanine, proline, tryptophan, tyrosine or valine. In preferred embodiments, the hydrophobic amino acid residues are selected from alanine, isoleucine, leucine, tryptophan, or valine residues, as exemplified by the peptides set forth in SEQ ID NOs: 2 to 8.

In one embodiment, the peptide is composed of His and a hydrophobic amino acid residue selected from Val, Leu, Ala, Trp, or Ile and may be amidated at the C-terminus, as exemplified by the 14-mer peptide of SEQ ID NO: 2 and the 15-mer peptides set forth in SEQ ID NOs: 3-4, in which five (one third) of the amino acid residues are D-amino acid residues. In another embodiment, the peptide is composed of His and Leu only and may be amidated at the C-terminus, as exemplified by the four 15-mer peptides of SEQ ID NOs: 5-8, in which 3 to 9 of the amino acid residues are D-amino acid residues.

The diastereomeric peptide of the invention may comprise one or more basic amino acid residues that may be derived from naturally or non-naturally occurring basic amino acids. In a preferred embodiment, the basic amino acid residues are selected from the lysine, arginine or ornithine residues, as exemplified by the peptides set forth in SEQ ID NOs: 9 to 26.

In one embodiment, the diastereomeric peptide is composed of histidine, lysine and the hydrophobic amino acid residues are leucine and isoleucine (SEQ ID NO: 9), or leucine and valine (SEQ ID NO: 10) or leucine, valine and tryptophan (SEQ ID NO: 11). In another preferred embodiment, the peptide is composed solely of histidine, leucine and lysine residues. Examples of such peptides are the 15-mer peptides of SEQ ID NOs: 12-17.

In another embodiment, the diastereomeric peptide of the invention is composed of histidine, lysine, arginine and one or more hydrophobic amino acid residues, for example, Leu and Val (SEQ ID NO: 18) or Ile, Leu and Val (SEQ ID NO: 19). More preferred diastereomeric peptides according to the invention are peptides composed solely of histidine, lysine, arginine and leucine. Examples of such peptides are the 15-mer and 16-mer peptides of SEQ ID NOs: 20 to 25. In another preferred embodiment, the diastereomeric peptide is composed solely of histidine, lysine, leucine and ornithine as exemplified by the peptide of SEQ ID NO:26.

In a further embodiment, the diastereomeric peptide of the invention may comprise, besides histidine, one or more hydrophobic amino acid residues and possibly one or more basic amino acid residues, an additional residue from a naturally or non-naturally occurring amino acid residue other than a hydrophobic or a basic amino acid residue. This additional amino acid may be located within the sequence of the peptide but, preferably, it is found at the N-terminus and/or C-terminus. When the amino acid is negatively charged such as aspartic acid or glutamic acid it is always located at the N-terminus or C-terminus. When the additional amino acid residue is asparagine, glutamine, glycine, serine, or threonine, it may be located within the sequence, but preferably will be at the N-terminus and/or C-terminus. Examples of such diastereomeric peptides are the peptides set forth in SEQ ID NOs: 27-33.

The additional amino acid may also be a non-natural amino acid. As used herein, the term “non-natural amino acid” for the additional or basic amino acid residue refers to modified natural α-amino acids such as chemical derivatives, e.g., hydroxyproline, α-carboxyglutamate, methionine sulfoxide, methionine methyl sulfonium and O-phosphoserine; N-alkyl, preferably N-methyl amino acids, e.g., N-methyl-valine, N-methyl-isoleucine, N-methyl-leucine, N-methyl-alanine; compounds in which a methylene residue was added to the amino acid backbone, e.g., homoserine, homoleucine, homoisoleucine, homolysine, or in which a methylene residue was deleted from the amino acid backbone, e.g., norvaline (Nva), norleucine (Nle); ornithine (2,5-diamino pentanoic acid), citrulline (2-amino-5-(carbamoylamino)pentanoic acid), diaminobutyric acid (DAB), 2-methyl-alanine. In one preferred embodiment, the non-natural amino acid is the basic amino acid ornithine.

In one embodiment of the invention, the diastereomeric peptide has at the N-terminus an additional amino acid such as glutamine, asparagine or threonine, as exemplified by the peptides of SEQ ID NOs: 27 to 30 and 31-33 or has at the C-terminus a glycine such as the peptide of SEQ ID NO: 31, or has a threonine at the N-terminus and a glycine at the C-terminus (SEQ ID NO: 32) or an asparagine at the N-terminus and a serine at the C-terminus (SEQ ID NO: 33).

In another embodiment, the diastereomeric peptide contains His, Leu and Lys residues and at the C-terminus an ornithine residue, such as the peptide of SEQ ID NO:27.

In still a further embodiment, the invention relates to cyclic derivatives of the diastereomeric peptide. The cyclic derivatives can be formed by methods known in the art. Examples of such cyclic peptides include the 17-mer peptides of the SEQ ID NOs: 34 and 35 containing His, Leu and Lys residues and two terminal cysteine residues for the formation of the cyclic derivative.

In yet a further embodiment, the diastereomeric peptide of the invention may be acylated at the N-terminus by an acyl group having at least 2 carbon atoms. The acyl group may be derived from an alkanoic acid of up to 6 carbon atoms and may be, for example, acetyl, propionyl, butyryl, pentanoyl, or hexanoyl, or an acyl group of a saturated or unsaturated fatty acid of at least 8 carbon atoms. Examples of fatty acids include octanoic acid, decanoic acid, undecanoic acid, dodecanoic acid, myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, and cerebronic acid.

In preferred embodiments, the peptide is acylated by an acetyl group as exemplified by the 15-mer Leu-His peptide of SEQ ID NO: 36, or by an hexanoyl group as exemplified by the 15-mer Leu-His peptide of SEQ ID NO: 37, or by an octanoyl group as exemplified by the 15-mer Leu-His peptide of SEQ ID NO: 38, or by a decanoyl group as exemplified by the 15-mer Leu-His peptide of SEQ ID NO: 39.

The peptides may also be substituted at the N-terminus by a hydrophobic C₂-C₂₀ alkyl or alkenyl and/or may be esterified or amidated at the C-terminus. The esters are preferably obtained by reaction with hydrophobic C₂-C₂₀, preferably C₅-C₁₈ fatty alcohols, and the amides are obtained by reaction with ammonia or with alkyl amines, wherein the alkyl is a C₂-C₂₀, preferably C₅-C₁₈, alkyl radical.

The term “C₂-C₂₀ alkyl” as used herein refers to a straight or branched alkyl radical having 2-20 carbon atoms and includes, for example, ethyl, n-propyl, iso-propyl, n-butyl, iso-butyl, tert-butyl, n-heptyl, n-hexyl, —C₁₀H₂₁, —C₁₅H₃₁, —C₁₆H₃₃, —C₁₇H₃₅, —C₁₈H₃₇, —C₂₀H₄₁, and the like. The term “C₂-C₂₀ alkenyl” refers to a straight or branched hydrocarbon radical having 2-20 carbon atoms and one or more double bonds, such as a terminal double bond, and includes, for example, vinyl, prop-2-en-1-yl, but-3-en-1-yl, pent-4-en-1-yl, hex-5-en-1-yl, —C₁₆H₃, C₁₈H₃₅.

In a further embodiment, the diastereomeric peptide of the invention comprises a hydrophobic amino-carboxylic acid moiety that may be linked covalently to the N-terminal amino acid, to the C-terminal amino acid, and/or to two amino acid residues within the sequence of the peptide via the α-amino of one amino acid residue and the α-carboxy of the other amino acid residue. The hydrophobic amino-carboxylic acid residue at the C-terminus of the peptide may be amidated.

The amino group of the hydrophobic amino-carboxylic acid may be any position of the molecule. In one embodiment, the hydrophobic amino-carboxylic acid is an α-amino-carboxylic acid of at least 4 carbon atoms such as, but not limited to, α-amino-hexanoic acid. An example of such a peptide is the acylated Leu-His diastereomeric peptide set forth in SEQ ID NO: 40, in which the α-amino-hexanoic acid moiety is inserted within the sequence between a His and a Leu residue.

In another preferred embodiment, the hydrophobic amino-carboxylic acid is an ω-amino-carboxylic acid of at least 4 carbon atoms such as, but not limited to, 4-amino-butyric acid, 6-amino-hexanoic acid, 8-amino-octanoic acid, 10-amino-decanoic acid, 12-amino-dodecanoic acid, 14-amino-myristic acid, 16-amino-palmitic acid, 18-amino-stearic acid, 18-amino-oleic acid, 16-amino-palmitoleic acid, 18-amino-linoleic acid, 18-amino-linolenic acid or 20-amino-arachidonic acid.

Preferred ω-amino-carboxylic acids according to the invention are 6-amino-hexanoic acid (6-amino-caproic acid) and 8-amino-octanoic acid (8-amino-caprylic acid). Examples of such peptides are the acylated Leu-His diastereomeric peptide set forth in SEQ ID NO: 41, in which the 8-amino-octanoic acid moiety is at the C-terminus and is amidated; the acylated Leu-His diastereomeric peptide set forth in SEQ ID NO: 42, in which the 6-amino-hexanoic acid moiety is inserted within the sequence between a His and a Leu residue; and the acylated Leu-His diastereomeric peptide set forth in SEQ ID NO: 43, in which the 8-amino-octanoic acid moiety is inserted within the sequence between a His and a Leu residue.

In another embodiment, the diastereomeric peptide may contain both an α-amino-carboxylic acid and an co-amino-carboxylic acid moiety. An example of such an embodiment is the Leu-His peptide of SEQ ID NO: 44, in which an α-amino-octanoic acid moiety is inserted within the sequence between a Leu and a His residue and an 8-amino-octanoic acid moiety is inserted between a his and a Leu moiety.

In a further embodiment of the invention, the diastereomeric peptide may be conjugated to a homing domain such as, but not limited to, a peptide comprising the integrin homing domain RGD or a hormone residue, as well known in the art.

As mentioned before, WO 2004/110341 of the same applicants discloses in a broad way acylated peptides that have a charge equal or greater than +1 and may contain histidine residues and D-amino acids. However, the peptides actually disclosed in said publication have at most 12 amino acids and are excluded from the present invention.

The above-mentioned WO 98/37090 of the same applicants discloses some sequences of diastereomeric peptides that comprise histidine and hydrophobic amino acids (see p. 46, peptides 67-72 and 79-80), but have never been synthesized and tested. These peptides of SEQ ID NOs: 45-52 are excluded from the present invention by the proviso in claim 1.

The diastereomeric peptides of the invention are useful for the treatment of cancer, both solid and non-solid tumor cancers and both primary tumors and metastases.

Examples of cancers that can be treated with the peptides of the invention include, but are not limited to, prostate cancer, bladder cancer, brain cancer, breast cancer, colorectal cancer, head and neck cancer, testicular cancer, ovarian cancer, pancreatic cancer, lung cancer, liver cancer, kidney cancer, gastrointestinal cancer, bone cancer, endocrine system cancers, lymphatic system cancers, melanoma, basal and squamous cell carcinomas, astrocytoma, pligodendroglioma, menigioma, neuroblastoma, glioblastoma, ependyoma, Schwannoma, neurofibrosarcoma, neuroblastoma, medullablastoma, fibrosarcoma, epidermoid carcinoma, skin cancer, and leukemia.

The present invention thus provides, in another aspect, a pharmaceutical composition comprising a diastereomeric peptide of the invention as defined hereinabove and a pharmaceutically acceptable carrier.

In one embodiment, the present invention provides pharmaceutical compositions comprising a diastereomeric peptide of the invention for the treatment of cancer. In preferred embodiments, the cancer is prostate cancer, breast cancer and metastases.

In one embodiment, the invention provides pharmaceutical compositions for topical application, for example for the treatment of topical infections caused by pathogenic organisms such as bacterial infections, particularly infections caused by bacteria resistant to antibiotics, and infections caused by pathogenic fungi. Examples of the use of such pharmaceutical compositions include topical treatment of: acne; topical infections caused by pathogenic organisms such as bacterial infections including chronic gastric mucosal infestation by Helicobacter pylori, intestinal bacterial infections, infections caused by antibiotic-resistant bacteria e.g. Streptococcus pyogenes and the methicilin-resistant Staphylococcus aureus; fungal infections including nail fungi, infections caused by yeasts such as Candida albicans, fungal infections of the scalp; fungal or bacterial infections related to surgical or traumatic wounds; chronic or poorly healing skin lesions such as foot ulcer in diabetes mellitus patients; vaginal infection (vaginitis); eye and ear infections; burn wounds; infections of mouth and throat; and localized infections such as chronic pulmonary infections in cystic fibrosis, emphysema and asthma.

The pharmaceutical composition may be in the form of solution, colloidal dispersion, cream, lotion, gel, foam, emulsion, spray, aerosol or other formulation for nasal or pulmonary application.

The diastereomeric peptides of the invention are effective against mycoplasma and can further be used to control/eliminate mycoplasma infection in cell cultures in a method comprising treating the cell culture with said diastereomeric peptide.

The invention thus also relates to a composition comprising a diastereomeric peptide of the invention to control mycoplasma infection in cell culture, for food preservation, or for use as food supplement.

In another embodiment, the invention relates to a veterinary composition comprising a diastereomeric peptide of the invention.

The invention further relates to the use of a diastereomeric peptide of the invention for the preparation of a pharmaceutical composition for topical application for treatment of bacterial or fungal infections, or of a pharmaceutical composition for the treatment of cancer.

The invention still further relates to a method for the treatment of an infection caused by a pathogenic organism that can be treated by topical application, which comprises administering topically to an individual in need thereof a diastereomeric peptide of the invention.

In another embodiment, the invention relates to a method for the treatment of a malignant tumor, which comprises administering to an individual in need thereof a therapeutically effective amount of a diastereomeric peptide of the invention.

### DETAILED DESCRIPTION OF THE INVENTION

The diastereomeric peptides of at least 13 amino acid residues of the present invention are characterized by comprising one or more histidine residues, thus differing from the 15-mer peptides disclosed in WO 02/040529. They have not been disclosed in the above-mentioned WO 98/37090 and WO 02/040529, and exhibit an enhanced or similar activity for the treatment of cancer in comparison to the closest diastereomer (peptide 4) disclosed in WO 02/040529.

The diastereomeric peptides of the invention are cytolytic agents of very low toxicity as evaluated herein in animal models. In the acute toxicity tests performed in mice, no mortality was observed with the peptides of the invention administered at concentrations considerably higher than those necessary for their anticancer activity, whereas 100% mortality was observed for the 15-mer peptide 4 of WO 02/040529, herein designated peptide 1 (SEQ ID NO: 1), administered at these high concentrations.

In order to reduce the toxicity and to improve the cytolytic activity of the diastereomeric peptide, the effect of several important parameters such as length, amphipathic organization, the variety of positively charged amino acids, the location and number of D-amino acids, additional amino acid residues at the N- and C-termini, and/or addition of hydrophobic chains to the N- and/or C-terminus, and polarity of the diastereomeric peptides, on their potency, selectivity and spectrum of activity were examined. For this purpose, we synthesized and structurally and functionally characterized a series of linear and cyclic diastereomers, basically comprised of various ratios of leucine and histidine and optionally containing lysine and arginine residues and additional amino acid residues, preferably at the N-terminus and/or C-terminus. The peptides were then characterized with regard to their biological activity towards pathogenic cancerous cells and normal mammalian cells such as NIH-3T3 normal fibroblasts cell line, and their toxicity was tested in vivo.

The potency and selectivity of the novel diastereomers of the invention is demonstrated herein in the anticancer assays. The diastereomers disclosed herein exhibit similar activity as that of peptide 1 disclosed in WO 02/040529 against several malignant cells and are more active against other cells. Furthermore, they are active against the malignant cells at concentrations that are 2-8 lower than the concentrations at which they act against NIH-3T3 normal fibroblasts cells. In addition, they are significantly less toxic to mice in comparison with peptide 1 of WO 02/040529.

Thus, the new diastereomeric peptides of the invention are useful as anticancer agents and can be used for treatment of solid tumors such as, but not limited to, breast, prostate, lung, kidney, and colon cancer as well as melanoma and basal and squamous cell carcinomas and non-solid tumors such as leukemias. The observed high potency of the positively charged diastereomeric peptides against a variety of malignant cells as shown in the examples herein indicates the existence of a common target for their action. This target is most probably the malignant cell membrane that has been shown to express higher levels of negatively charged phosphatidylserine than normal mammalian cells (Utsugi et al., 1991).

In one preferred embodiment, the present invention provides pharmaceutical compositions comprising a diastereomeric peptide of the invention for the treatment of cancer. It is contemplated that all peptides of the invention are useful for the treatment of malignant tumors as shown herein for peptides of SEQ ID NOs: 5, 8, 12-16, 36-39, 42 and 44, designated in the examples herein as peptides 5, 8, 12-16, 36-39 42 and 44, respectively. In particular, peptides 12, 13 and 37-39, 42 and 44 were shown in experiments in vitro to be effective against prostate tumor.

The high potency and the low in-vivo toxicity of the model diastereomers of the invention pave the way for their use also in topical applications against a wide variety of pathogenic organisms in topical infections including, but not limited to, treatment of acne, fungal infections of the scalp, fungal or bacterial infections related to surgical or traumatic wounds, chronic or poorly healing skin lesions (especially in diabetics), vaginal infection (vaginitis), eye and ear infections and burn wounds, infections of mouth and throat, and localized infections such as chronic pulmonary infections in cystic fibrosis, emphysema or asthma that can be treated with aerosol or other formulation for nasal or pulmonary application. The observed resistance of the diastereomers to proteolytic digestion may enable them to reach the digestive system in intact form and to eliminate there bacterial infections such as chronic gastric mucosal infestation by Helicobacter pylori and intestinal bacterial infections. As used herein, the term “topical” means “pertaining to a particular surface area” and the topical agent applied to a certain area of said surface will affect only the area to which it is applied. Therefore, any and all applications in which the peptides act locally and not through the blood circulation are encompassed by the present invention.

For systemic administration, the peptide may be administered as such without any additional carrier or, in general, buffered aqueous compositions are employed. Alternate compositions utilize liposome carriers. The solution is buffered at a desirable pH using conventional buffers such as Hank's solution, Ringer's solution, or phosphate buffer. Other components which do not interfere with the activity of the peptide may also be included such as stabilizing amounts of proteins, for example, serum albumin, or low density- or high density-lipoprotein (LDL and HDL, respectively).

Systemic formulations can be administered by injection, such as intravenous (i.v.), intraperitoneal (i.p.), intramuscular, or subcutaneous (s.c.) injection, or can be administered by transmembrane or transdermal techniques.

For topical application, the active components can be formulated with a variety of cosmetically and/or pharmaceutically acceptable carriers. Formulations appropriate for transdermal or transmembrane administration include sprays and suppositories containing skin penetrants, which can often be detergents.

The term “pharmaceutically acceptable carrier” refers to a vehicle that delivers the active components to the intended target without being harmful to humans or other recipient organisms. As used herein, “pharmaceutical” will be understood to encompass both human and animal pharmaceuticals. Useful carriers include, for example, water, acetone, ethanol, ethylene glycol, propylene glycol, butane-1,3-diol, isopropyl myristate, isopropyl palmitate, or mineral oil. The carrier may be in any form appropriate to the mode of delivery, for example, solutions, colloidal dispersions, emulsions (oil-in-water or water-in-oil), suspensions, creams, lotions, gels, foams, mousses, sprays and the like. Methodology and components for formulation of pharmaceutical compositions are well known, and can be found, for example, in Remington's Pharmaceutical Sciences, Eighteenth Edition, A. R. Gennaro, Ed., Mack Publishing Co. Easton Pa., 1990.

The formulation, in addition to the carrier and the anticancer components, also can comprise other optional materials that may be chosen depending on the carrier and/or the intended use of the formulation. Additional components include, but are not limited to, antioxidants, chelating agents, emulsion stabilizers, e.g. carbomer, preservatives, e.g. methyl paraben, fragrances, humectants, e.g. glycerine, waterproofing agents, e.g. PVP/Eicosene copolymer, water soluble film-formers, e.g. hydroxypropyl methylcellulose, oil-soluble film formers, cationic or anionic polymers, and the like.

The diastereomers of the invention may also be used for food preservation, as food supplements in veterinary compositions, as alternative to antibiotics for animal nutrition, as anti-mycoplasma, antibacterial, and antifungal agents for tissue culture media, and as reagents for transformation/transfection of target cells with desired DNA or RNA molecules.

The invention will now be described with reference to the following non-limiting examples.

### EXAMPLES

### Materials and Methods

### (i) Materials

4-Methyl benzhydrylamine resin (BHA) and butyloxycarbonyl (Boc) amino acids were purchased from Calbiochem-Novabiochem Co. (La Jolla, Calif., USA). Other reagents used for peptide synthesis included trifluoroacetic acid (TFA, Sigma), N,N-diisopropylethylamine (DIEA, Sigma), dicyclohexylcarbodiimide (DCC, Fluka), 1-hydroxybenzotriazole (1-HOBT, Pierce), and dimethylformamide (DMF, peptide synthesis grade, Biolab, Ill.).XTT reaction solution for cytotoxicity assay and matrigel were purchased from Biological Industries (Beit Haemek, Israel). All other reagents were of analytical grade. Buffers were prepared in double-distilled water.

### (ii) Cell Culture

The CL1 human prostate carcinoma (PC) cell line is an androgen-independent (AI) subclone of LNCaP cell line, which was generated by culturing androgen-dependent (AD) LNCaP cells in charcoal-stripped, AD serum, as described (Patel et al. 2000). 22RV1 human PC cells are AI sub-clones of the AD prostatic adenocarcinoma CWR22 xenograft (Sramkoski et al. 1999). CL1 and 22RV1 (ATCC, USA) were grown in RPMI-1640 supplemented with 10% FCS (Biological Industries, Beit Haemek, Israel). PC3 and DU145 are androgen-insensitive (AI) (non-responsive), invasive human prostate cancer cell lines. NIH-3T3 mouse fibroblast cell lines (ATCC, USA) were grown in DMEM supplemented with 10% BS. Murine Lewis lung carcinoma (LLC) cell lines were also grown in DMEM medium supplemented with 10% fetal calf serum and antibiotics under the same conditions as above.

(iii) Peptide Synthesis and Purification

Peptides were synthesized by a solid phase method on 4-methyl benzhydrylamine resin (BHA) (0.05 meq) (Merrifield et. al., 1982; Shai et. al., 1990). The resin-bound peptides were cleaved from the resin by hydrogen fluoride (HF) and after HF evaporation and washing with dry ether, the peptides were extracted with 50% acetonitrile/water. HF cleavage of the peptides bound to BHA resin resulted in C-terminus amidated peptides. Each crude peptide contained one major peak, as revealed by RP-HPLC (reverse phase high-performance liquid chromatography) that was 60-80% pure peptide by weight. The synthesized peptides were further purified by RP-HPLC on a C₁₈ (Supleco) reverse phase Bio-Rad semi-preparative column (250×10 mm, 300 m pore size, 5-μm particle size), in 30 min, using a linear gradient of 30-50% acetonitrile in water, both containing 0.1% TFA (v/v), at a flow rate of 1.5 [1.8] ml/min. The purified peptides, which were shown to be homogeneous (˜95%) by analytical HPLC, were subjected to amino acid analysis and electrospray mass spectroscopy to confirm their composition and molecular weight.

N-Acylation was carried out using the same protocol used to attach protected amino acids for peptide synthesis.

### (iv) Synthesis of Cyclic Diastereomers.

The cyclic peptides were synthesized by a solid-phase method as described in section (iii) above, with or without cysteine residues at both the N- and C-termini of the peptides. The cyclization without cystein is carried out by protecting the N-terminal, activating the C-terminal, then deprotecting the N-terminal and reacting the C- and N-terminal groups while still bound to the resin. When the peptide contains cystein residues at both the N- and C-termini, after HF cleavage and RP-HPLC purification, the peptides are solubilized at low concentration in PBS (pH 7.3), and cyclization is completed after 12 h. The cyclic peptides are further purified on RP-HPLC and subjected to amino acid analysis to confirm their composition, and SDS-PAGE to confirm their monomeric state.

### Example 1

### Synthesis of His-Containing Diastereomeric Peptides

The following 15-mer peptide 1 and 13-17-mer C-amidated diastereomeric peptides 2-44 (SEQ ID Nos 2-44) composed of His, one or more hydrophobic amino acids selected from Leu, Ile, Val, Ala, Thr and Trp, or another non-natural hydrophobic amino acid, optionally the positively charged amino acids Lys, His and/or Arg and/or the N-cap amino acids Gln and Asn, and optionally further acylated at the N-terminus, containing from 3 to 9 D-amino acid residues, were synthesized as described in Material and Methods, sections (iii) and the cyclic peptides 34 and 35 are prepared as described in section (iv). Peptide 1 is a 15-mer diastereomer described in the above-mentioned WO 02/040529 and herein used for comparison purposes. The peptides will be represented hereinafter by numerals in bold and by a sequence identity number (SEQ ID NO.).

The bold and underlined amino acids are D-amino acids.

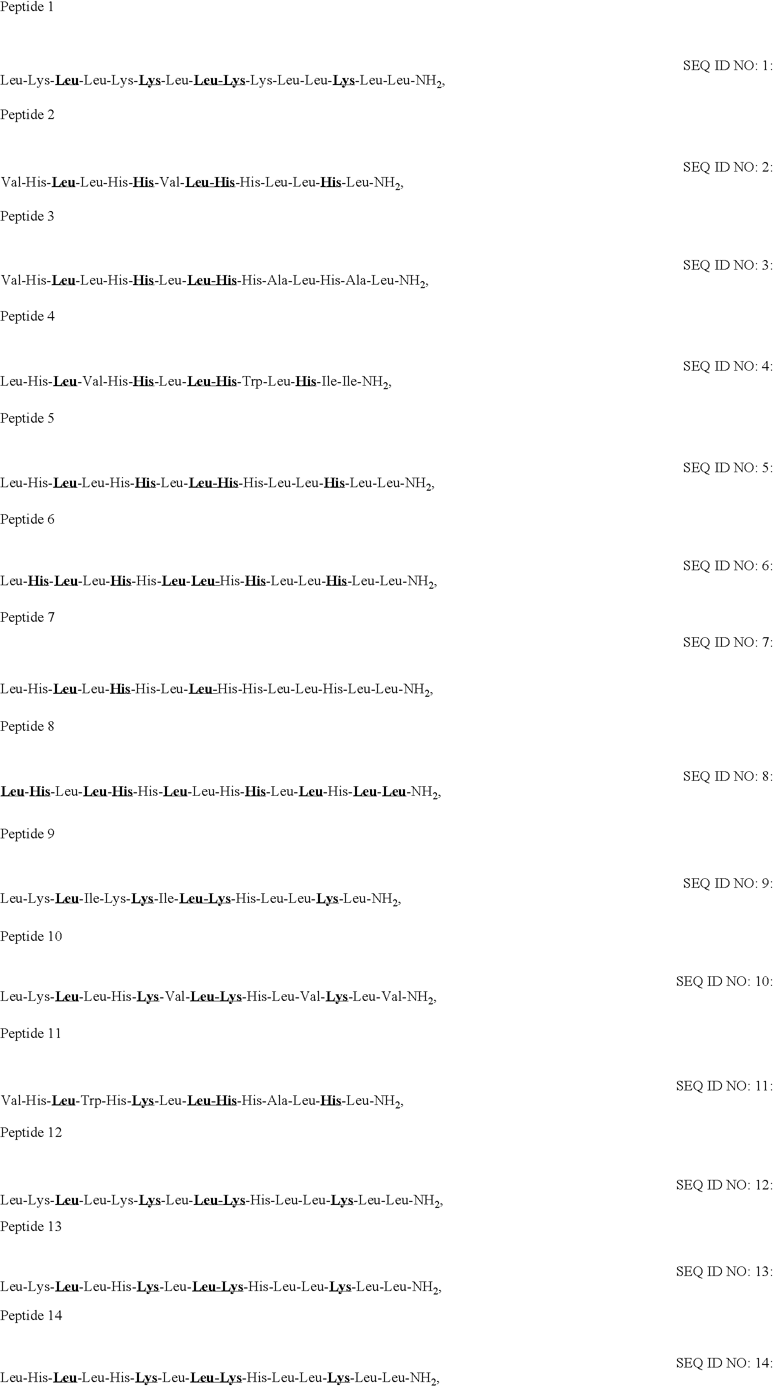

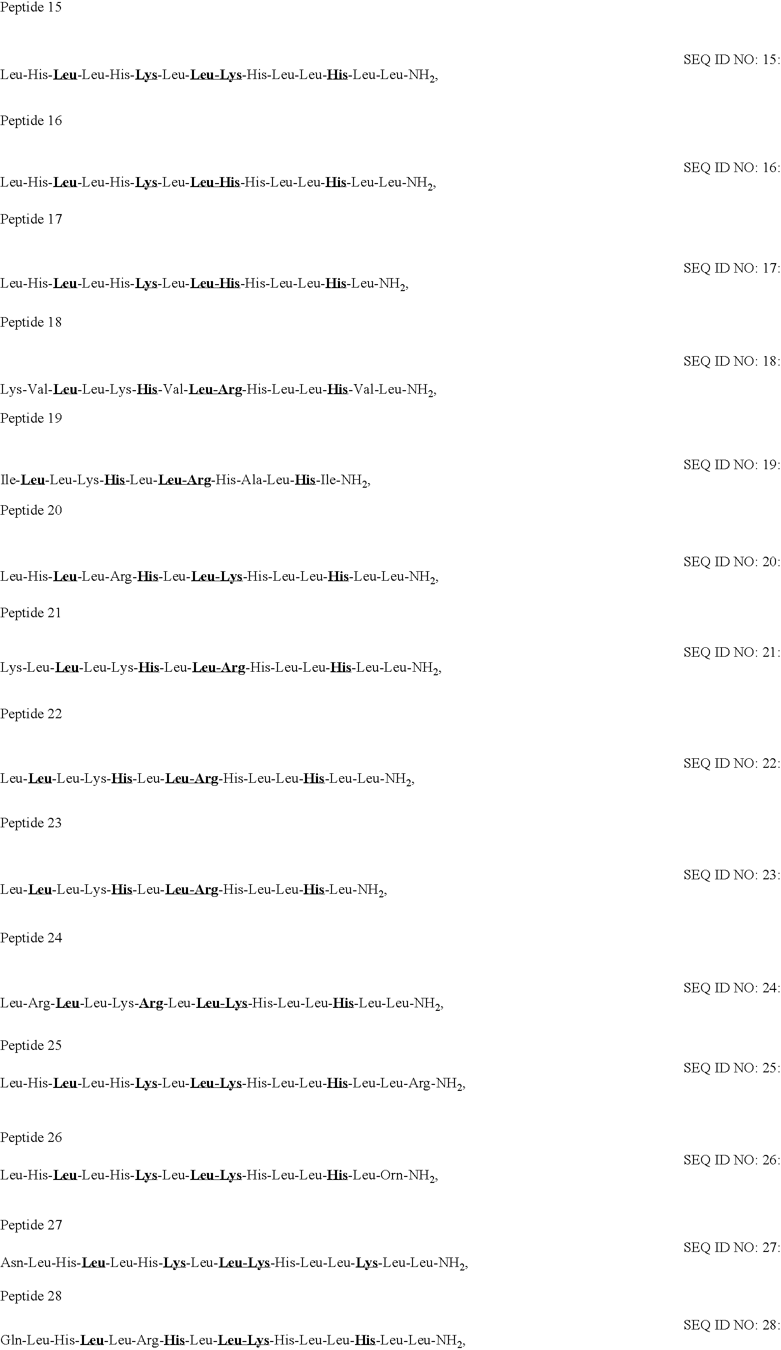

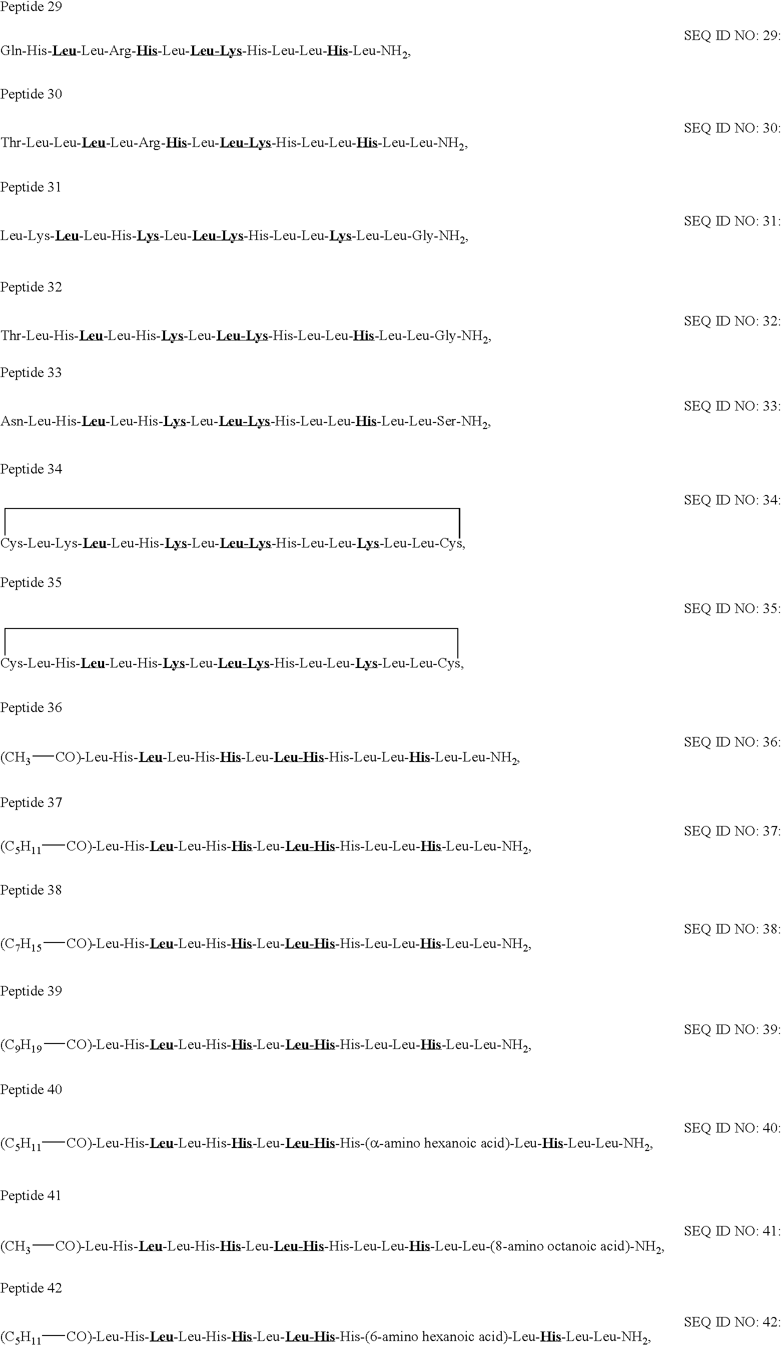

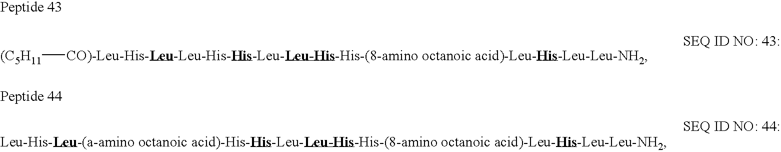

As representative examples, the analysis data of peptides 13 and 1 are given. Peptide 13 was obtained as a white powder of ≧98% purity as determined by HPLC. Amino acids content: His-2, Leu-9 and Lys-3.85. Molecular weight by Mass spectra analysis: 1822.5. Peptide 1 was obtained as a white powder of ≧99% purity and molecular weight 1804.5. Amino acids content: Leu-9 and Lys-5.80.

### Example 2

### Cytotoxicity Assay (XTT Proliferation Assay)

The anticancer activity of the diastereomers 1, 5 and 11-16, 36-39, 42 and 44 was examined against human CL1 prostate cancer, murine LLC (Lewis lung carcinoma), DU 145 and PC3 cell lines. The cell selectivity of the diastereomeric peptides was also studied by examining their effect on NIH-3T3 normal mouse fibroblasts cell line.

A 96-well plate (Falcon) was used for the XTT proliferation assay. Cancer cells were grown for 24 hours (day 1) in RPMI-1640 medium (5×10³, 7×10³, 1×10⁴ and 7×10³ cells/100 μl for LLC, CL1, 22RVI, DU 145 and PC3, respectively) supplemented with 10% fetal calf serum and antibiotics, at 37° C., in humidified atmosphere at 5% CO₂ and 95% air, resulting in growth medium pH of 7.4. NIH-3T3 fibroblast cells (1×10⁴ cells/100 μl) were grown in DMEM medium supplemented with 10% bovine calf serum and antibiotics under the same conditions as described above for the cancerous cells. Wells in the last two rows served as blanks (medium only, for measuring the background color of the medium) and 100% survival controls (cells and medium only without treatment), respectively.

In day 2, the peptides were dissolved in sterile PBS to a concentration of 200 μM (or 500 μM). The medium in the assay wells was replaced with 100 μl serum free medium. For the assays at pH 6, the medium was initially concentrated (×5 or ×10) and then diluted with double distilled water and addition of sodium carbonate. Before reaching the correct dilution, the medium was adjusted to pH 6 and then more water was added to reach the final dilution. The cells were grown in physiological pH, and the acidic pH was used only during the 24 hours incubation with the peptides. A sign for the correct acidity was a light yellow color of the medium. In line A of the plate, 160 μl of serum free medium was added, if the initial peptide concentration was 500 μM.

Peptide solutions, 100 μl (or 40 μl) were added to each assay well in line A, such that the final concentration of the peptide was 100 mM and the volume 200 μl. The medium in the wells was mixed with multichannel pipette 5 times and 100 μl of it were transferred to the next row of wells (line B), to give a peptide concentration of 50 μM. The double dilution of the peptides continued downstream the plate in the same manner. The plates were then incubated for 24 h.

In day 3, XTT reaction solution was prepared by adding to 100 μl aliquots of activation solution (sodium 3′-[1-(phenyl-aminocarbonyl)-3,4-tetrazolium]-bis(4-methoxy-6-nitro) benzene sulfonic acid hydrate and N-methyl dibenzopyrazine methyl sulfate; mixed in a proportion of 50:1) (protected from light and kept in −20° C.), the substrate. 50 μl of XTT reaction solution were added to each well and the plates were incubated for 2 hours (37° C. and 5% CO₂+95% air. In cases the incubation was not enough for the creation of the color, it was extended for up to 24 hours). The optical density was read at wavelength of 450 nm in an ELISA plate reader. Cell viability was determined relative to the control and final results were recorded. The results were confirmed using replications in at least three independent experiments. The LC50 for each peptide was obtained from the curve of cell viability versus concentration of peptide and taken from the concentration at which cell viability was 50%. The data shown in Table 1 are for only one experiment, but representative of all replications.

With regard to human cells, the results for peptides 5, 8 and 12-16 show that the diastereomers 12 and 13 of the invention are similarly or significantly more potent than the known 15-mer diastereomer 1 against the human CL1 cell line, at pH 6.0, and peptide 12 was more active than peptide 1 against LLC at both pH 6 and pH 7.4. Furthermore, peptides 8, 12, 13, 14, 16, 36, 42 and 44, were active against CL1 and LLC at pH 7.4 at concentrations, which are 2-8 fold lower than the concentration at which they act against NIH-3T3 normal mouse fibroblast cells. At pH 7.4, peptide 14 was active against CL1 at concentrations 8-fold lower than the concentrations at which it was active against NIH-3T3 cells, although it was half as active against CL1 and 22RVI as peptide 1. The diastereomers 5, 8, 12, 13, 14, 16, 36-39, 42 and 44 were significantly more potent against 22 RVI cells at pH 6 than peptide 1, particularly peptides 39 and 44 were 8 and 16 times more active compared to 1. In addition, peptides 5, 14 and 16 were active against CL1 cells at concentrations at least two-fold lower than the concentrations at which they act against NIH-3T3 cells at the same pH. Particularly high activity was observed for 38, 39, 42 and 44 at pH 6 against 22RVI and LLC cells as compared to NIH3T3 cells.

Peptides 10 and 11 were significantly less potent against DU 145 cells at both pH 7.4 and 6, and against PC3 cells at pH 7.4 compared to their activity against other cells.

These results clearly reveal that the new diastereomeric peptides of the invention are more selective and more effective than the known diastereomer 1.

TABLE 1
Lethal Concentration (LC₅₀) (μg/ml) of peptides 1, 5, 8, 12-16, 36-39, 42 and 44
against CL1, 22RVI, LLC, DU145 and PC3 cancer cells and normal fibroblast
cell lines

| | pH 7.4 | | | | | | pH 6 | | | | |
|---|---|---|---|---|---|---|---|---|---|---|---|
| Pep. | NIH3T3 | CL1 | 22RV1 | LLC | DU145 | PC3 | NIH3T3 | CL1 | 22RV1 | LLC | DU145 |
| 1 | 50 | 6.25 | 12.5 | 12.5 | 4.7 | 6.2 | 25 | 9.3 | 25 | 12.5 | 4.7 |
| 12 | 50 | 6.25 | 12.5 | 6.25 | ND | ND | 12.5 | 6.25 | 12.5 | 6.25 | ND |
| 13 | 50 | 6.25 | 12.5 | 12.5 | 25 | 25 | 19 | 6.25 | 12.5 | 12.5 | 18.7 |
| 14 | 100 | 12.5 | 25 | 50 | 12.5 | 18.7 | 25 | 12.5 | 12.5 | 25 | 9.3 |
| 15 | ND | ND | ND | 50 | ND | ND | 25 | 12.5 | ND | ND | ND |
| 16 | 100 | 12.5 | ND | 50 | ND | ND | 25 | ND | 12.5 | ND | ND |
| 5 | 100 | 12.5 | ND | ND | ND | ND | 50 | 12.5 | 12.5 | ND | ND |
| 36 | >100 | 12.5 | >100 | 50 | ND | ND | 12.5 | 6.25 | 12.5 | 12.5 | ND |
| 37 | >100 | 12.5 | >100 | >100 | ND | ND | 100 | 3.12 | 6.25 | 50 | ND |
| 38 | >100 | 6.25 | 100 | 100 | ND | ND | >100 | 1.56 | 6.25 | 100 | ND |
| 39 | >100 | 6.25 | 100 | 100 | ND | ND | >100 | <0.78 | 1.56 | 100 | ND |
| 42 | >100 | 6.25 | 100 | 50 | ND | ND | >100 | 6.25 | 6.25 | 50 | ND |
| 44 | 100 | 3.25 | 100 | 50 | ND | ND | 100 | 1.56 | 3.12 | 50 | ND |
| 8 | 50 | 12.5 | 12.5 | 25 | ND | ND | 25 | 12.5 | 12.5 | 12.5 | ND |

Pep. = Peptide
ND—not determined
PC3 cells did not grow at pH 6

### Example 3

### Acute Toxicity Test in Mice

Acute toxicity of peptides 1, 13 and 16 was examined by intravenously injecting mice (n=3), each with one dose per day for 2 days of 0.5 ml solution containing peptides 1, 13 or 16 at 3, 9, 15, 20 and 30 mg/kg of body weight. No mortality was observed with all the peptides administered at 3 mg/kg and 9 mg/kg. At 15 and 20 mg/kg of body weight, 100% mortality was observed only with prior art peptide 1 and no mortality was observed with peptides 13 and 16 of the invention. No mortality was observed with peptide 16 of the invention at 30 mg/kg.

A week after injecting the peptides, blood samples were taken from the survived mice. All the differential and biochemistry tests were in the range of normal values (i.e. neutrophils, lymphocytes, monocytes, eosinophils, basophiles, creatine phosphokinase, alkaline phosphatase, alanine aminotransferase, aspartate aminotransferase and creatinine). Thus, the peptides 13 and 16 of the invention are not toxic even when administered at concentrations considerably higher than those necessary for their anticancer activity.

### Example 4

### Anticancer Activity of the Peptides In Vivo

(i) Inhibition of tumor growth in human prostate cancer xenografts. Subcutaneouse (s.c.) implantation of human PC cells in mice was done as previously described in Gavish et al. (Gavish et al. 2002). Briefly, 0.1 ml AI CL1 or 22RV1 human PC cells (5×10⁶ cells) in Matrigel were inoculated s.c. into the dorsal side of five to six week-old nude male mice weighing 20-25 g (Harlen Co., Israel). Two weeks after cell implantation, when the tumors diameter reached ≧5 mm (denoted as day 1), the diastereomer 13 and its all L-amino acid analog peptide (at 1 mg/kg, 0.1 mM), or vehicle (PBS, pH=7.4) were injected intratumorally (dosing volume of 2.5 ml/kg) three times a week for a total of 9 doses. Tumor size was measured by a caliper and recorded twice a week during a period of 28 days. Mice were weighed and tumor weight (mg) was estimated by using the formula of length×width×depth×0.52 in mm³, assuming the specific gravity to be 1. At the end of the treatment, the mice were killed, and the tumors were removed, photographed, and weighed. The animal experimentation was conducted according to the rules of the Institutional Animal Care and Use Committee.

Serum PSA levels. Four weeks after the first treatment, blood was withdrawn from the 22RV1-inoculated mice in order to determine the level of prostate specific antigen (PSA). The blood samples were taken directly to heparin containing tubes, centrifuged, and the supernatants were stored at −20° C. The CanAg PSA EIA kit (CanAg Diagnostics) was used to determine the total PSA in the mice plasma (Gavish et al., 2002). Tumor weight and PSA levels, represented as the mean±SE, were calculated from the raw data and then subjected to Student's t test. A value of P<0.05 was considered as statistically significant.

Independently of the xenograft type, a significant reduction in tumor weight was observed with the mice treated with peptide 13 but not in mice treated with the analog L-diastereomer. In some mice, the tumor completely disappeared. Furthermore, in the PSA-secreting 22RV1 xenografts, the reduction in tumor weight was accompanied by a marked decrease in the PSA serum levels. The treatment with peptide 13 showed an increase in the body weight of the animals compared with the vehicle-treated control group. In contrast, the L-diastereomer was inactive in both xenograt models.

To check the reason why all L-amino acid analog was not active as opposed to the diastereomer peptide 13, both peptides were mixed with Matrigel matrix for one hour and the solution was analyzed by using RP-HPLC and mass spectroscopy. Upon interaction with the Matrigel matrix, the all L-amino acid analog peptide was fully inactivated in contrast to peptide 13, which preserved ˜50% of its activity.

### (ii) Inhibition of Prostate Tumor-Derived Lung Metastases Formation

Since 22RV1 prostate tumor is metastatic, we analyzed the ability of the systemically administered peptide 13 to inhibit the formation of lung metastases derived from prostate cancer in CD1 nude mice that were pre-injected systemically with cells. During the experiment, the mice were monitored continuously for clinical signs of toxicity. It was observed that throughout the assay period the animals that had been treated with peptide 13 were in good condition and did not express any sign of weakness. At the end of the experiment, we found that the lung metastases were entirely abolished in the peptide 13 treated animals as compared to the untreated controls. Moreover, the peptide 13 treated mice also showed a significant increase in the body weight compared with the vehicle treated control group.

(iii) Inhibition of Tumor Growth in Breast Cancer Xenografts

RFP-MDA-MB-231 breast cancer (BC) cells were injected (5×10⁶ cells in 0.1 ml PBS) into the left mammary fat pad of 8-week-old female SCID/NCr mice (NCI, USA) as previously described (Dadiani et al., 2004). One week after cell implantation, when the tumor diameter reached ˜5 mm, peptide 13 (at 5 mg/kg, 0.14 mM), or vehicle (PBS, pH=7.4) was injected systemically (dosing volume of 22 ml/kg) three times a week for a total of 9 doses for ten mice. Mice were weighed and tumor volume was measured by a caliper (expressed in weight units (mg) (Papo et al., 2003) twice a week for a period of 45 days.

Monitoring of solid breast tumor and its derived metastases was done by in vivo fluorescence. Tumor fluorescence intensity was monitored in real time by using in vivo optical imaging system (IVIS) and was recorded once a week during the period of 38 days.

A major reduction in tumor size was recorded from caliper measurements. The reduction in tumor size was accompanied by a marked lowering of the tumor fluorescence as recorded from in vivo optical imaging by IVIS. However, since the accuracy and sensitivity of the fluorescence detection was much greater then caliper measurements, a lowering of tumor fluorescence was observed much sooner. The treatment with peptide 13 also resulted in an increase in the body weight of the animals compared with the vehicle-treated control group

Animals treated with peptide 13 were in good condition throughout the assay period and did not express any signs of weakness.

(iv) Inhibition of Formation of Lung and Lymph Node Metastases Derived from Breast Cancer

Since MDA-MB-231 breast tumor cells were metastatic, the ability of the systemically administered peptide 13 to inhibit the formation of metastases in the lymph nodes and lungs of SCID/NCr mice was analyzed. During the experiment, the mice were monitored continuously for clinical signs of toxicity.

Monitoring of solid metastases derived for breast tumor was done by in vivo fluorescence using IVIS as described above in (iii). At the end of the treatment (day 38), the mice were killed, and the lungs and lymph nodes were removed and monitored for fluorescence of metastases derived from the breast cancer. For metastases quantification, the lungs and right lymph nodes were excised and fixed in 4% buffered formaldehyde. Paraffin-embedded 5-μm sections were stained with H&E. The percentage of metastatic cell area of total section area was calculated using the Image-Pro plus 4.1 software.

A significant reduction in lymph node metastasis fluorescence intensity was observed in the mice after treatment with peptide 13. Images of dissected lungs and lymph nodes from the untreated mice were also analyzed showing strong fluorescence relative to the treated ones.

The dissected lungs and lymph nodes were analyzed by histology. The lungs and lymph nodes in the control untreated mice were significantly more populated by the cancer cells while the tumors in the 15-mer treated mice were much less densely populated. Metastasis quantification was done according to areas from three different sections of each organ (P<0.005).

### Example 5

### Resistance of the Diastereomers to Proteolytic Digestion

In order to reach their target, the diastereomers have to withstand proteolytic digestion of proteases, which may occur after their administration and during the time until they reach the target site. The susceptibility of the peptides 13 and 14 to proteolytic digestion by pepsin (from porcine stomach mucosa, Sigma), trypsin (from bovine pancreas, Sigma), and elastase (from human leukocytes, Sigma) was assessed by reverse-phase HPLC. As a negative control, the all L-amino acid analog peptides were used. Equal amounts of the peptides were dissolved in PBS (35 mM phosphate buffer/0.15 M NaCl, pH 7.3) at a final concentration of 140 μM, to which 25 μM of protease were added. The samples were incubated under agitation for 30 min at 37° C. After the addition of the appropriate protease inhibitor to stop the reaction, aliquots were injected to C₁₈ column and the amounts of the intact peptides 13 and 14 and their all L analogs were evaluated using their absorbance at 215 nm. The diastereomers of the invention were significantly less susceptible to proteases digestion (˜50% after 2 hr) whereas the all L analogs were completely degraded after 30 min.

### Example 6

### Liposome Encapsulation of the Diastereomeric Peptides

Liposomes serve as convenient delivery vehicles for biologically active molecules. Hydrophilic drugs can be encapsulated in the internal aqueous compartment, whereas hydrophobic drugs may bind to or are incorporated in the lipid bilayers. In this experiment, liposomal diastereomeric peptides were prepared in order to further lower the peptide toxicity and increase their selectivity.

Liposomes composed of different ratios of phosphatidylcholine (PC)/phosphatidylglycerol (PG) (9:1; 4:1; 1:1 w/w) or phosphatidylethanolamine (PE)/PG (9:1; 4: 1; 1:1 w/w) were prepared. Briefly, dry lipid mixtures were dissolved in CHCl₃/MeOH (2/1, v/v). The solvents were evaporated under nitrogen stream, and the lipid mixtures at the compositions described above were resuspended in PBS by vortex mixing. The lipid suspension was extruded through 3 different polycarbonate filters (1 μm, 0.2 μm and 0.1 μm pore size filters, 15 times each). Finally, the resulting suspensions of large unilamellar vesicles (LUV) were added to different concentrations of a peptide of the invention to give lipid/peptide ratios of 50:1; 30:1; 10:1 w/w, respectively. The mixtures were sonicated for 2 minutes and the liposomes were stored at 4° C. until used.

The anticancer activity of the resulting liposomal diastereomeric peptide preparations was examined as described in Example 2 above. The LC₅₀ of liposomal peptides 5 and 12-16 in various lipid compositions and peptide/lipid ratios (as described above), or of liposomes at lipid compositions equivalent to the loaded liposomes or peptides alone, were determined using LC1, LLC and 22RVI cell lines. Liposomal peptides exhibited LC₅₀ results similar to those of the peptide alone, indicating that the peptides of the invention entrapped within liposomes can maintain their anticancer activity. However, this activity is dependent on the liposomes' lipid composition and on the lipid/peptide ratio.

Based on the in vitro test results, the in vivo toxicity of the liposomal peptides preparations was examined, utilizing the liposomal composition which gave the best anticancer activity. Groups of 5 CD1 male mice weighing 24-27 g (5-week old), bred in an animal isolator (IVC racks) under specific pathogen-free (SPF) conditions at 24±1° C., were used. Twelve mg/kg liposomal peptide or peptide alone dissolved in PBS in a dosing volume of 10 ml/kg, were administered by single i.v. bolus injection via the mice tail vein. In parallel, control groups received i.v. injections of equivalent liposomes alone or PBS in a dosing volume of 10 ml/kg.

The liposomal peptides maintained their activity but were less toxic, thus leading to reduced mortality in mice. No incidence of mortality occurred following the i.v. injection of PBS or the liposomes alone.

### REFERENCES

Avrahami D. and Shai Y. A new group of antifungal and antibacterial lipopeptides derived from non-membrane active peptides conjugated to palmitic acid. J. Biol. Chem., 279:12277-12285, 2004.
Avrahami D. and Shai Y. Bestowing antifungal and antibacterial activities by lipophilic acid conjugation to D,L-amino acid-containing antimicrobial peptides: A plausible mode of action. Biochem., 42: 14946-14956, 2003.
Baker, M. A., Maloy, W. L., Zasloff, M., and Jacob, L. S. Anticancer efficacy of Magainin 2 and analogue peptides. Cancer Res., 53: 3052-3057, 1993.
Biragyn, A., Surenhu, M., Yang, D., Ruffini, P. A., Haines, B. A., Klyushnenkova, E., Oppenheim, J. J., and Kwak, L. W. Mediators of innate immunity that target immature, but not mature, dendritic cells induce antitumor immunity when genetically fused with nonimmunogenic tumor antigens. J. Immunol., 167: 6644-6653, 2001.
Boman, H. G. Peptide antibiotics and their role in innate immunity. Annu. Rev. Immunol., 13: 61-92, 1995.
Chan, S. C., Yau, W. L., Wang, W., Smith, D. K., Sheu, F. S., and Chen, H. M. Microscopic observations of the different morphological changes caused by anti-bacterial peptides on Klebsiella pneumoniae and HL-60 leukemia cells. J. Pept. Sci., 4: 413-425, 1998.
Chen, Y., Xu, X., Hong, S., Chen, J., Liu, N., Underhill, C. B., Creswell, K., and Zhang, L. RGD-Tachyplesin inhibits tumor growth. Cancer Res., 61: 2434-2438, 2001.
Dadiani, M., Margalit, R., Sela, N., and Degani, H. High-resolution magnetic resonance imaging of disparities in the transcapillary transfer rates in orthotopically inoculated invasive breast tumors. Cancer Res. 64:3155-3161, 2004.
Draize J. H., Woodward, G. & Calvery, H. O. Methods for the study of irritation and toxicity of substances applied topically to the skin and mucous membranes. J Pharmacol Exp Ther 82: 377-390, 1944.
Ellerby, H. M., Arap, W., Ellerby, L. M., Kain, R., Andrusiak, R., R10, G. D., Krajewski, S., Lombardo, C. R., Rao, R., Ruoslahti, E., Bredesen, D. E., and Pasqualini, R. Anti-cancer activity of targeted pro-apoptotic peptides. Nat. Med., 5: 1032-1038, 1999.
Gavish, Z., Pinthus, J. H., Barak, V., Ramon, J., Nagler, A., Eshhar, Z., and Pines, M. Growth inhibition of prostate cancer xenografts by halofuginone. The Prostate, 51: 73-83, 2002.
Hong, J., Oren, Z. & Shai, Y. Structure and organization of hemolytic and nonhemolytic diastereomers of antimicrobial peptides in membranes. Biochemistry, 38: 16963-16973, 1999.
Hui, L., Leung, K., and Chen, H. M. The combined effects of antibacterial peptide cecropin A and anti-cancer agents on leukemia cells. Anticancer Res., 22: 2811-2816, 2002.
Jacob, E., Schopperle, K. and Bredt, W. Adherence inhibition assay: a specific serological test for detection of antibodies to Mycoplasma pneumoniae. Eur. J. Clin. Microbiol. 4: 113-118, 1985.
Leuschner, C., Enright, F. M., Gawronska, B., and Hansel, W. Membrane disrupting lytic peptide conjugates destroy hormone dependent and independent breast cancer cells in vitro and in vivo. Breast Cancer Res. Treat., 78: 17-27, 2003.
Mai, J. C., Mi, Z., Kim, S. H., Ng, B., and Robbins, P. D. A proapoptotic peptide for the treatment of solid tumors. Cancer Res., 61: 7709-7712, 2001.
Malina A. and Shai Y. Conjugation of fatty acids with different lengths modulates the antibacterial and antifungal activity of a cationic biologically inactive peptide. Biochem J., 390: 695-702, 2005.
Oppenheim, J. J., Biragyn, A., Kwak, L. W., and Yang, D. Roles of antimicrobial peptides such as defensins in innate and adaptive immunity. Ann. Rheum. Dis., 62: ii17-21, 2003.
Oren, Z. and Shai, Y. Selective Lysis of Bacteria but not Mammalian Cells by Diastereomers of Melittin: Structure-Function Study. Biochemistry, 36: 1826-1835, 1996.
Oren, Z. and Shai, Y. Cyclization of a cytolytic amphipathic alpha-helical peptide and its diastereomer: effect on structure, interaction with model membranes, and biological function. Biochemistry 39: 6103-6114, 2000.
Oren, Z., Hong, J. and Shai, Y. A repertoire of novel antibacterial diastereomeric peptides with selective cytolytic activity. J. Biol. Chem. 272: 14643-14649, 1997.
Papo, N. and Shai, Y. New Lytic Peptides Based on the D, L Amphipathic Helix Motif Preferentially Kill Tumor Cells Compared to Nornal Cells. Biochemistry, 42: 9346-9354, 2003.
Papo, N., Shahar, M., Eisenbach, L., and Shai, Y. A novel lytic peptide composed of D, L amino acids selectively kills cancer cells in culture and in mice. J. Biol. Chem., 278: 21018-21023, 2003.
Park, Y., Lee, D. G., Jang, S. H., Woo, E. R., Jeong, H. G., Choi, C. H., and Hahm, K. S. A Leu-Lys-rich antimicrobial peptide: activity and mechanism. Biochim. Biophys. Acta, 21: 172-182, 2003.
Patel, B. J., Pantuck, A. J., Zisman, A., Tsui, K. H., Paik, S. H., Caliliw, R., Sheriff, S., Wu, L., deKernion, J. B., Tso, C. L., and Belldegrun, A. S. CL1-GFP: an androgen independent metastatic tumor model for prostate cancer. J. Urol., 164: 1420-1425, 2000.
Porgador, A., Bannerji, R., Watanabe, Y., Feldman, M., Gilboa, E. & Eisenbach, L. Antimetastatic vaccination of tumor-bearing mice with two types of IFN-gamma gene-inserted tumor cells. J. Immunol. 150: 1458-1470, 1993.
Raff, M. C. Surface antigenic markers for distinguishing T and B lymphocytes in mice. Transpl. Rev. 6: 52-80, 1971.
Shai, Y. & Oren, Z. Diastereomers of Cytolysins, a Novel Class of Potent Antibacterial Peptides J. Biol. Chem. 271: 7305-7308, 1996.
Sharon, M., Oren, Z., Shai, Y. & Anglister, J. 2D-NMR and ATR-FTIR study of the structure of a cell-selective diastereomer of melittin and its orientation in phospholipids. Biochemistry 38: 15305-15316, 1999.
Shin, S. Y., Lee, S. H., Yang, S. T., Park, E. J., Lee, D. G., Lee, M. K., Eom, S. H., Song, W. K., Kim, Y., Hahm, K. S., and Kim, J. I. Antibacterial, antitumor and hemolytic activities of alpha-helical antibiotic peptide, P18 and its analogs. J. Pept. Res., 58: 504-514, 2001.
Sramkoski, R. M., Pretlow, T. G., 2nd, Giaconia, J. M., Pretlow, T. P., Schwartz, S., Sy, M. S., Marengo, S. R., Rhim, J. S., Zhang, D., and Jacobberger, J. W. A new human prostate carcinoma cell line, 22Rv1. In Vitro Cell Dev. Biol. Anim., 35: 403-409, 1999.
Utsugi, T., Schroit, A. J., Connor, J., Bucana, C. D. & Fidler, I. J. Elevated expression of phosphatidylserine in the outer membrane leaflet of human tumor cells and recognition by activated human blood monocytes. Cancer Res. 51: 3062-3066, 1991.
Wang, Z. and Wang, G. APD: the Antimicrobial Peptide Database. Nucleic Acids Res., 32: D590-592, 2004.
Warren, P., Li, L., Song, W., Holle, E., Wei, Y., Wagner, T., and Yu, X. In vitro targeted killing of prostate tumor cells by a synthetic amoebapore helix 3 peptide modified with two gamma-linked glutamate residues at the COOH terminus. Cancer Res., 61: 6783-6787, 2001.
Yang, D., Biragyn, A., Kwak, L. W., and Oppenheim, J. J. Mammalian defensins in immunity: more than just microbicidal. Trends Immunol., 23: 291-296, 2002.

1-47. (canceled)

48. A diastereomeric peptide with a net positive charge greater than +1, and cyclic derivatives thereof, having at least 13 amino acid residues, comprising histidine and one or more hydrophobic amino acid residues, optionally esterified or amidated at the C-terminus and/or acylated at the N-terminus, excluding the peptides set forth in SEQ ID Nos: 45-52.

49. The diastereomeric peptide according to claim 48, wherein said one or more hydrophobic amino acid residues are from naturally or non-naturally occurring hydrophobic amino acids.

50. The diastereomeric peptide according to claim 49, wherein said one or more hydrophobic amino acid residues are selected from the group consisting of naturally occurring α-amino acids such as alanine, cysteine, isoleucine, leucine, methionine, phenylalanine, proline, tryptophan, tyrosine or valine, preferably alanine, isoleucine, leucine, tryptophan, or valine residues.

51. The diastereomeric peptide according to claim 50, selected from the group consisting of the peptides set forth in SEQ ID NOs: 2 to 8.

52. The diastereomeric peptide according to claim 48, comprising one or more basic amino acid residues selected from lysine, arginine and/or ornithine residues.

53. The diastereomeric peptide according to claim 52, selected from the group consisting of the peptides set forth in SEQ ID NOs: 9 to 12 and 15 to 26.

54. The diastereomeric peptide according to claim 48, having a net positive charge greater than +1 and 15 amino acid residues, comprising histidine, leucine and lysine, optionally esterified or amidated at the C-terminus and/or acylated at the N-terminus.

55. The diastereomeric peptide according to claim 54, selected from the peptides set forth in SEQ ID NOs: 13 and 14.

56. The diastereomeric peptide according to claim 48, comprising a naturally or non-naturally occurring amino acid residue other than a hydrophobic or a basic amino acid residue, preferably at the N-terminus and/or C-terminus.

57. The diastereomeric peptide according to claim 56, wherein said amino acid residue is aspartic acid or glutamic acid at the N-terminus or C-terminus, or asparagine, glutamine, glycine, serine, or threonine at the N-terminus and/or C-terminus.

58. The diastereomeric peptide according to claim 57, selected from the group consisting of the peptides set forth in SEQ ID NOs: 27-33.

59. A diastereomeric peptide according to claim 48, wherein said peptide is cyclic.

60. The diastereomeric peptide according to claim 59, wherein said cyclic peptide is selected from the group consisting of the peptides set forth in SEQ ID NOs: 34-35.

61. The diastereomeric peptide according to claim 48, wherein said peptide is acylated at the N-terminus by an acyl group having at least 2 carbon atoms, such as acetyl, propionyl, butyryl, pentanoyl, hexanoyl or an acyl group of a saturated or unsaturated fatty acid of at least 8 carbon atoms, such as octanoic acid, decanoic acid, undecanoic acid, dodecanoic acid, myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, or cerebronic acid.

62. The diastereomeric peptide according to claim 61, wherein said acylated peptide is selected from the group consisting of the peptides set forth in SEQ ID NOs: 36-39.

63. The diastereomeric peptide according to claim 48, comprising a hydrophobic amino-carboxylic acid moiety linked covalently to the N-terminal amino acid, to the C-terminal amino acid, and/or to two amino acid residues within the sequence of the peptide via the α-amino of one amino acid residue and the α-carboxy of the other amino acid residue.

64. The diastereomeric peptide according to claim 63, comprising:
(i) an α-amino-carboxylic acid of at least 4 carbon atoms, preferably α-amino-hexanoic acid linked preferably to the C-terminus of the peptide;
(ii) an ω-amino-carboxylic acid of at least 4 carbon atoms selected from the group consisting of 4-amino-butyric acid, 6-amino-hexanoic acid, 8-amino-octanoic acid, 10-amino-decanoic acid, 12-amino-dodecanoic acid, 14-amino-myristic acid, 16-amino-palmitic acid, 18-amino-stearic acid, 18-amino-oleic acid, 16-amino-palmitoleic acid, 18-amino-linoleic acid, 18-amino-linolenic acid or 20-amino-arachidonic acid, preferably 6-amino-hexanoic acid or 8-amino-octanoic acid; or
(iii) both α-amino-carboxylic acid and ω-amino-carboxylic acid moieties of at least 4 carbon atoms, preferably α-amino-octanoic acid and 8-amino-octanoic acid.

65. The diastereomeric peptide according to claim 64, selected from the group consisting of: (i) the peptide set forth in SEQ ID NO: 40; (ii) the peptides set forth in SEQ ID NO: 41 to 43; or (iii) the peptide set forth in SEQ ID NO: 44.

66. The diastereomeric peptide according to claim 48, conjugated to a homing domain selected from a peptide comprising the integrin homing domain RGD or a hormone residue.

67. The diastereomeric peptide according to claim 48, having 13, 14, 15 or 16 amino acid residues.

68. A pharmaceutical composition comprising a diastereomeric peptide according to claim 48 and a pharmaceutically acceptable carrier.

69. The pharmaceutical composition according to claim 68, in the form of solution, colloidal dispersion, cream, lotion, gel, foam, emulsion, spray, aerosol or other formulation for nasal or pulmonary application.

70. The pharmaceutical composition according to claim 68, for topical application.

71. A method for treating cancer comprising administering to a subject in need thereof a therapeutically effective amount of a diastereomeric peptide according to claim 48.

72. The method according to claim 71, wherein the cancer is selected from the group consisting of solid and non-solid tumors, primary tumors or metastases.

73. The method according to claim 71, wherein said cancer is selected from the group consisting of prostate cancer, bladder cancer, brain cancer, breast cancer, colorectal cancer, head and neck cancer, testicular cancer, ovarian cancer, pancreatic cancer, lung cancer, liver cancer, kidney cancer, gastrointestinal cancer, bone cancer, endocrine system cancers, lymphatic system cancers, melanoma, basal and squamous cell carcinomas, astrocytoma, pligodendroglioma, menigioma, neuroblastoma, glioblastoma, ependyoma, Schwannoma, neurofibrosarcoma, neuroblastoma, medullablastoma, fibrosarcoma, epidermoid carcinoma, skin cancer, or leukemia.

74. A method for treating an infection comprising administering to a subject in need thereof a therapeutically effective amount of a diastereomeric peptide according to claim 48.

75. The method according to claim 74, comprising topical treatment of bacterial or fungal infections, selected from the groups consisting of: acne; topical infections caused by pathogenic organisms such as bacterial infections including chronic gastric mucosal infestation by Helicobacter pylori, intestinal bacterial infections, infections caused by antibiotic-resistant bacteria e.g. Streptococcus pyogenes and the methicilin-resistant Staphylococcus aureus; fungal infections including nail fungi, infections caused by yeasts such as Candida albicans, fungal infections of the scalp; fungal or bacterial infections related to surgical or traumatic wounds; chronic or poorly healing skin lesions such as foot ulcer in diabetes mellitus patients; vaginal infection (vaginitis); eye and ear infections; burn wounds; infections of mouth and throat; and localized infections such as chronic pulmonary infections in cystic fibrosis, emphysema and asthma.

76. A composition comprising a diastereomeric peptide according to claim 48, to control mycoplasma infection in cell culture, for food preservation, or for use as food supplement.

77. A veterinary composition comprising a diastereomeric peptide according to claim 48.


SEQUENCE LISTING (52 sequences)

SEQ ID NO: 1 - protein, 15 aa, Artificial
  Synthetic
LKLLKKLLKKLLKLL

SEQ ID NO: 2 - protein, 14 aa, Artificial
  Synthetic
VHLLHHVLHHLLHL

SEQ ID NO: 3 - protein, 15 aa, Artificial
  Synthetic
VHLLHHLLHHALHAL

SEQ ID NO: 4 - protein, 15 aa, Artificial
  Synthetic
LHLVHHLLHHWLHII

SEQ ID NO: 5 - protein, 15 aa, Artificial
  Synthetic
LHLLHHLLHHLLHLL

SEQ ID NO: 6 - protein, 15 aa, Artificial
  Synthetic
LHLLHHLLHHLLHLL

SEQ ID NO: 7 - protein, 15 aa, Artificial
  Synthetic
LHLLHHLLHHLLHLL

SEQ ID NO: 8 - protein, 15 aa, Artificial
  Synthetic
LHLLHHLLHHLLHLL

SEQ ID NO: 9 - protein, 14 aa, Artificial
  Synthetic
LKLIKKILKHLLKL

SEQ ID NO: 10 - protein, 15 aa, Artificial
  Synthetic
LKLLHKVLKHLVKLV

SEQ ID NO: 11 - protein, 14 aa, Artificial
  Synthetic
VHLWHKLLHHALHL

SEQ ID NO: 12 - protein, 15 aa, Artificial
  Synthetic
LKLLKKLLKHLLKLL

SEQ ID NO: 13 - protein, 15 aa, Artificial
  Synthetic
LKLLHKLLKHLLKLL

SEQ ID NO: 14 - protein, 15 aa, Artificial
  Synthetic
LHLLHKLLKHLLKLL

SEQ ID NO: 15 - protein, 15 aa, Artificial
  Synthetic
LHLLHKLLKHLLHLL

SEQ ID NO: 16 - protein, 15 aa, Artificial
  Synthetic
LHLLHKLLHHLLHLL

SEQ ID NO: 17 - protein, 14 aa, Artificial
  Synthetic
LHLLHKLLHHLLHL

SEQ ID NO: 18 - protein, 15 aa, Artificial
  Synthetic
KVLLKHVLRHLLHVL

SEQ ID NO: 19 - protein, 13 aa, Artificial
  Synthetic
ILLKHLLRHALHI

SEQ ID NO: 20 - protein, 15 aa, Artificial
  Synthetic
LHLLRHLLKHLLHLL

SEQ ID NO: 21 - protein, 15 aa, Artificial
  Synthetic
KLLLKHLLRHLLHLL

SEQ ID NO: 22 - protein, 14 aa, Artificial
  Synthetic
LLLKHLLRHLLHLL

SEQ ID NO: 23 - protein, 13 aa, Artificial
  Synthetic
LLLKHLLRHLLHL

SEQ ID NO: 24 - protein, 15 aa, Artificial
  Synthetic
LRLLKRLLKHLLHLL

SEQ ID NO: 25 - protein, 16 aa, Artificial
  Synthetic
LHLLHKLLKHLLHLLR

SEQ ID NO: 26 - protein, 15 aa, Artificial
  Synthetic
LHLLHKLLKHLLHLX

SEQ ID NO: 27 - protein, 16 aa, Artificial
  Synthetic
NLHLLHKLLKHLLKLL

SEQ ID NO: 28 - protein, 16 aa, Artificial
  Synthetic
QLHLLRHLLKHLLHLL

SEQ ID NO: 29 - protein, 14 aa, Artificial
  Synthetic
QHLLRHLLKHLLHL

SEQ ID NO: 30 - protein, 16 aa, Artificial
  Synthetic
TLLLLRHLLKHLLHLL

SEQ ID NO: 31 - protein, 16 aa, Artificial
  Synthetic
LKLLHKLLKHLLKLLG

SEQ ID NO: 32 - protein, 17 aa, Artificial
  Synthetic
TLHLLHKLLKHLLHLLG

SEQ ID NO: 33 - protein, 17 aa, Artificial
  Synthetic
NLHLLHKLLKHLLHLLS

SEQ ID NO: 34 - protein, 17 aa, Artificial
  Synthetic
CLKLLHKLLKHLLKLLC

SEQ ID NO: 35 - protein, 17 aa, Artificial
  Synthetic
CLHLLHKLLKHLLKLLC

SEQ ID NO: 36 - protein, 15 aa, Artificial
  Synthetic
LHLLHHLLHHLLHLL

SEQ ID NO: 37 - protein, 15 aa, Artificial
  Synthetic
XHLLHHLLHHLLHLL

SEQ ID NO: 38 - protein, 15 aa, Artificial
  Synthetic
XHLLHHLLHHLLHLL

SEQ ID NO: 39 - protein, 15 aa, Artificial
  Synthetic
XHLLHHLLHHLLHLL

SEQ ID NO: 40 - protein, 15 aa, Artificial
  Synthetic
XHLLHHLLHHXLHLL

SEQ ID NO: 41 - protein, 16 aa, Artificial
  Synthetic
LHLLHHLLHHLLHLLX

SEQ ID NO: 42 - protein, 15 aa, Artificial
  Synthetic
XHLLHHLLHHXLHLL

SEQ ID NO: 43 - protein, 15 aa, Artificial
  Synthetic
XHLLHHLLHHXLHLL

SEQ ID NO: 44 - protein, 15 aa, Artificial
  Synthetic
LHLXHHLLHHXLHLL

SEQ ID NO: 45 - protein, 16 aa, Artificial
  Synthetic
RIIVHIRLRIIHHIRL

SEQ ID NO: 46 - protein, 16 aa, Artificial
  Synthetic
HIGIKAHVRIIRVHII

SEQ ID NO: 47 - protein, 16 aa, Artificial
  Synthetic
RIYVKIHLRYIKKIRL

SEQ ID NO: 48 - protein, 16 aa, Artificial
  Synthetic
KIGHKARVHIIRYKII

SEQ ID NO: 49 - protein, 16 aa, Artificial
  Synthetic
RIYVKPHPRYIKKIRL

SEQ ID NO: 50 - protein, 16 aa, Artificial
  Synthetic
KPGHKARPHIIRYKII

SEQ ID NO: 51 - protein, 16 aa, Artificial
  Synthetic
RIYVKPHPRYIKKIRL

SEQ ID NO: 52 - protein, 16 aa, Artificial
  Synthetic
KPGHKARPHIIRYKII